(19) United States
(12) Patent Application Publication
(10) Pub. No.: US 2014/0316287 A1
(43) Pub. Date: Oct. 23, 2014

(54) System and method for displaying fluid responsivenss predictors

(71) Applicants: COVIDIEN LP, Mansfield, MA (US); COVIDIEN LP, Boulder, CO (US)

(72) Inventors: James Nicholas Watson, Dunfermline (GB); Paul Stanley Addison, Edinburgh (GB)

(73) Assignees: COVIDIEN LP, Mansfield, MA (US); COVIDIEN LP, Boulder, CO (US)

(21) Appl. No.: 14/255,061

(22) Filed: Apr. 17, 2014

Related U.S. Application Data
(60) Provisional application No. 61/815,104, filed on Apr. 23, 2013.
(60) Provisional application No. 61/815,412, filed on Apr. 24, 2013.

(51) Int. Cl. A61B 5/00; A61B 5/022; A61B 5/0215; A61B 5/029; A61B 5/0295

(52) U.S. Cl. CPC A61B 5/7275; A61B 5/029; A61B 5/0295; A61B 5/0215; A61B 5/02233; A61B 5/742
USPC 600/485; 600/481; 600/526; 600/507

(57) ABSTRACT

Embodiments provide systems and methods for displaying a fluid responsiveness predictor (FRP) based on an analysis a physiological signal detected by a physiological sensor applied to a patient. A method may include detecting the signal of the patient with the physiological sensor, determining an FRP with a FRP determination module, wherein the determining operation comprises analyzing at least one characteristic of the physiological signal over time to determine the FRP, receiving a report request to report the FRP at a requested time through a user interface, generating a reported FRP in relation to the requested time using the FRP determination module, and displaying the reported FRP on a display. The displaying operation may include displaying the FRP using at least one graphic representation.

### CROSS-REFERENCE TO RELATED APPLICATIONS

The present application relates to and claims priority benefits from U.S. Provisional Patent Application No. 61/815,104, entitled “System and Method for Displaying Fluid Responsiveness Parameters,” filed Apr. 23, 2013, which is hereby expressly incorporated by reference in its entirety.

The present application also relates to and claims priority benefits from U.S. Provisional Patent Application No. 61/815,412, entitled “System and Method for Displaying Fluid Responsiveness Parameters,” filed Apr. 24, 2013, which is hereby expressly incorporated by reference in its entirety.

### FIELD OF THE DISCLOSURE

Embodiments of the present disclosure generally relate to physiological signal processing and, more particularly, to systems and methods of displaying at least one fluid responsiveness predictor determined through analysis of at least one physiological signal output by at least one sensor operatively connected to a patient.

### BACKGROUND OF THE DISCLOSURE

Fluid responsiveness represents a prediction of whether fluid loading will improve blood flow within a patient. Fluid responsiveness refers to the response of stroke volume or cardiac output to fluid administration. A patient is said to be fluid responsive if fluid loading accomplishes improved blood flow, such as by an improvement in cardiac output or stroke volume index by about 10%, 15% or more. Fluid is delivered with the expectation that it will increase the patient's cardiac preload, stroke volume, and cardiac output, resulting in improved oxygen delivery to the organs and tissue. Fluid delivery may also be referred to as volume expansion, fluid therapy, fluid challenge, or fluid loading. Monitoring fluid responsiveness allows a physician to determine whether additional fluid should be provided to an individual, such as through an intravenous fluid injection.

Various dynamic measures have been proposed for determining the fluid responsiveness of a patient. A number of fluid responsiveness predictors (FRPs) utilize the variation in the amplitude of a physiological signal over the respiratory cycle, such as stroke volume variation (SVV), pulse pressure variation (PPV), and variations in the amplitude of the cardiac pulses of a plethysmographic (PPG) signal, such as a pulse oximetry signal. Often, however, a clinician may not fully understand and/or be confident in an output FRP. For example, a typical output FRP may simply be a numerical value. Accordingly, while the clinician may see the numerical value of the PPV, he/she may not witness an associated change in stroke volume, for example.

Further, an FRP may be continuously determined and output throughout a time that a patient is monitored. For example, the FRP may be computed over a certain analysis time window (e.g. over a rolling 60 second time window), or over a previous number of breaths (e.g. 3 breaths). The calculated latest value of the FRP may then be used to update the reported value on the device once per reporting update period, which may be less than the analysis time window (e.g., a reporting update period of every 5 seconds).

However, during periods of poor signal quality or localized changes in physiological conditions (e.g., during posture changes, drug administration, etc.), the reported FRP may contain a significant error and/or be unrepresentative of the actual FRP that describes the fluid responsiveness of the patient. In addition, the reported parameter may rely solely on a detected physiological signal and no other information source, thereby causing variability in the quality and value of the reported rate over time, and thus the value reported to the clinician. Accordingly, the value of the FRP used by the clinician may depend upon the time at which the clinician observed the monitored value.

### SUMMARY OF EMBODIMENTS OF THE PRESENT DISCLOSURE

Certain embodiments of the present disclosure provide a method for displaying a fluid responsiveness predictor (FRP) based on an analysis of one or more physiological signals detected by a physiological sensor applied to a patient. The method may include detecting the physiological signal(s) of the patient with the physiological sensor, and determining an FRP with a FRP determination module. The determining operation may include analyzing at least one characteristic of the physiological signal(s) over time to determine the FRP, inputting a report request to report the FRP at a requested time through a user interface, generating a reported FRP in relation to the requested time using the FRP determination module, and displaying the reported FRP on a display from the requested time until one of a cease report instruction input through the user interface or a predefined end time for a reported FRP. The displaying operation may include displaying the FRP using at least one graphic representation (in which the graphic representation is other than a text or numeric value displayed on a screen). The method may also include refraining from displaying the FRP if the user request is not input. The reported FRP may be based on a considered time period that extends from an initial time to at least the requested time.

While the FRP may be continuously determined by the FRP determination module, the FRP may not be reported (for example, shown as a numerical value or graphic representation). The FRP may be reported (that is, the reported FRP) at the request of an individual.

In at least one embodiment, the generating the reported FRP operation may include refraining from considering noise within the physiological signal(s). The noise may be generated through patient motion (for example, posture changes, coughing, or the like), drug/medication administration, etc. In at least one embodiment, the generating the reported FRP operation may include generating an average of the FRP over a considered time period that extends from an initial time to at least the requested time. The initial time may be a time before the requested time For example, the initial time may be 5, 10, 15, or 20 minutes before the requested time. Alternatively, the initial time may be less than 5 minutes before the request time, or more than 20 minutes before the requested time.

In at least one embodiment, the graphic representation(s) may include a difference bracket that includes an upper line extending from a maximum peak value of a portion of the physiological signal(s), a lower line extending from a minimum peak value of the portion of the physiological signal(s), and a difference line extending between the upper line and the lower line. In at least one other embodiment, the graphic representation(s) may include a shaded or colored area between a maximum peak value of a portion of the physiological signal(s) and a minimum peak value of the portion of the physiological signal(s). In at least one other embodiment the graphic representation(s) may include a minimum band related to a minimum peak value of the physiological signal(s), and a maximum band related to a maximum peak value of the physiological signal(s). In at least one other embodiment, the graphic representation(s) may include a minimum peak value of the one or more physiological signals superimposed on a maximum peak value of the one or more physiological signals. The graphic representation(s) may include at least one shape indicating the reported FRP.

The method may also include inputting patient information after the inputting the report request operation, and adjusting one or both of the analyzing or determining operations based on the patient information. The patient information may include height, weight, body mass index (BMI), body surface area (BSA), hydration level, skin pigmentation, medication information, and/or the like.

The physiological signal(s) may include, for example, at least one blood pressure signal, at least one plethysmographic (PPG) signal, or at least one stroke volume signal.

Certain embodiments of the present disclosure provide a system for displaying a fluid responsiveness predictor (FRP) based on an analysis of one or more physiological signals of a patient. The system may include a physiological sensor configured to detect the physiological signal(s) of the patient, a FRP determination module configured to determine the FRP through an analysis of at least one characteristic of the physiological signal(s) over time, a user interface configured to allow a user to input a report request to report the FRP at a requested time, an FRP reporting module configured to receive the report request and instruct the FRP determination module to generate a reported FRP in relation to the requested time, and an FRP display module configured to display the reported FRP on a display from the requested time until one of a cease report instruction input through the user interface or a predefined end time for the reported FRP. The reported FRP may be displayed having at least one graphic representation.

Certain embodiments of the present disclosure provide a system for displaying a fluid responsiveness predictor (FRP) based on an analysis of one or more physiological signals of a patient. The system may include a physiological sensor configured to detect the physiological signal(s) of the patient, a user interface configured to allow a user to input a report request to report the FRP at a requested time, and at least one FRP processor configured to: (a) determine the FRP through an analysis of at least one characteristic of the physiological signal(s) over time (b) receive the report request and generate a reported FRP in relation to the requested time, and (c) display the reported FRP on a display from the requested time until one of a cease report instruction input through the user interface or a predefined end time for the reported FRP. The reported FRP may be displayed having at least one graphic representation.

Certain embodiments of the present disclosure provide a method for graphically displaying a predictor of fluid responsiveness of a subject. The method may include receiving a physiological signal representative of a blood flow characteristic of the subject, calculating a fluid responsiveness predictor based on modulations of the physiological signal, and displaying a graphical indication of the fluid responsiveness predictor. The graphical indication includes a representation of an area between portions of the physiological signal. The portions of the physiological signal may be, for example, maximum and minimum peaks, waveforms, curves, or the like.

### BRIEF DESCRIPTION OF THE DRAWINGS

FIG. 1 illustrates a block diagram of a system for displaying a fluid responsiveness predictor, according to an embodiment of the present disclosure.

FIG. 2 illustrates a block diagram of a system for displaying a fluid responsiveness predictor, according to an embodiment of the present disclosure.

FIG. 3 illustrates a display showing a graphic representation of a fluid responsiveness predictor, according to an embodiment of the present disclosure.

FIG. 4 illustrates a display showing a graphic representation of a fluid responsiveness predictor, according to an embodiment of the present disclosure.

FIG. 5 illustrates a display showing a graphic representation of a fluid responsiveness predictor, according to an embodiment of the present disclosure.

FIG. 6A illustrates a display showing a graphic representation of a fluid responsiveness predictor, according to an embodiment of the present disclosure.

FIG. 6B illustrates a display showing a graphic representation of a fluid responsiveness predictor, according to an embodiment of the present disclosure.

FIG. 7 illustrates a front view of a monitoring device, according to an embodiment of the present disclosure.

FIG. 8 illustrates a representation of a PPG signal, according to an embodiment of the present disclosure.

FIG. 9 illustrates a display showing a graphic representation of a fluid responsiveness predictor, according to an embodiment.

FIG. 10 illustrates a display showing a graphic representation of a fluid responsiveness predictor, according to an embodiment.

FIG. 11 illustrates a physiological signal over time, according to an embodiment of the present disclosure.

FIG. 12 illustrates a flow chart of a process of displaying a fluid responsiveness predictor, according to an embodiment of the present disclosure.

FIG. 13 illustrates a perspective view of a monitoring system, according to an embodiment of the present disclosure.

Before the embodiments of the disclosure are explained in detail, it is to be understood that the disclosure is not limited in its application to the details of construction and the arrangement of the components set forth in the following description or illustrated in the drawings. The disclosure is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION OF THE DRAWINGS

FIG. 1 illustrates a block diagram of a system 10 for displaying a fluid responsiveness parameter or predictor (FRP), according to an embodiment of the present disclosure. The system 10 may include a physiological sensor 12 operatively connected to a patient 14, and a monitoring device 16 communicatively connected to the physiological sensor 12. The monitoring device 16 may include an FRP determination module 18 in communication with an FRP display module 20, a display 22, an FRP reporting module 24, and a user interface 26.

The physiological sensor 12 is configured to sense or detect at least one physiological signal of the patient 14. For example, in at least one embodiment, the physiological sensor 12 may be or include a blood pressure detection device, such as an invasive arterial line (“A-line”) that may be positioned within the vasculature of the patient, or a non-invasive blood pressure cuff that may be positioned around a portion of patient anatomy, such as an arm. The blood pressure detection device may sense the physiological signal in the form of a blood pressure signal of the patient 14.

In at least one other embodiment, the physiological sensor 12 may be or include a plethysmographic (PPG) sensor, such as a pulse oximetry sensor, that may be positioned on a finger, forehead, forearm, or the like of the patient 14. The PPG sensor is configured to detect a PPG signal, which may be in the form of a PPG waveform, responsive to the blood flow of the individual. The PPG signal is a non-invasive, optical measurement that may be used to detect changes in blood volume within tissue, such as skin, of an individual. In general, the PPG signal is a physiological signal that includes an AC physiological component related to cardiac synchronous changes in the blood volume with each heartbeat. The PPG signal also includes a DC baseline component that may be related to respiration, sympathetic nervous system activity, and thermoregulation. The PPG signal may be analyzed to determine physiological characteristics such as respiration rate, respiratory effort, pulse rate, oxygen saturation, and/or the like.

In at least one other embodiment, the physiological sensor 12 may be or include an echocardiography sensing device that may be positioned over a chest of the patient 14. The echocardiography sensing device may be configured to detect a physiological signal in the form of a stroke volume signal of the patient.

The monitoring device 16 is operatively connected to the physiological sensor 12, such as through a wired or wireless connection. The monitoring device 16 may be or include a personal computer, laptop computer, workstation, smart device, such as a handheld tablet or smart phone, and/or the like. The physiological sensor 12 detects or senses the physiological signal and outputs the physiological signal to the monitoring device 16. The FRP determination module 18 receives the physiological signal and analyzes one or more characteristics, features, parameters, aspects, or components of the physiological signal over time to determine the FRP.

For example, the FRP determination module 18 may analyze a blood pressure signal of the patient 14 to determine a pulse pressure variation (PPV) of the blood pressure signal over time. The PPV may be used as the FRP. In at least one other embodiment, the FRP determination module 18 may analyze a PPG signal of the patient 14 to determine at least one respiratory variation of the PPG signal over time. The respiratory variation(s) of the PPG signal may be used as the FRP. In at least one other embodiment, the FRP determination module 18 may analyze a stroke volume signal of the patient 14 to determine a stroke volume variation (SW) of the stroke volume signal over time. The SW may be used as the FRP. It is to be understood that the PPV, variation(s) in the PPG signal, and SW are merely examples of FRPs. The FRP determination module 18 may analyze one or more physiological signals to determine various other FRPs.

After the FRP determination module 18 determines the FRP, the FRP display module 20 displays a graphic representation 28 of the FRP on the display 22, which may be or include a monitor, screen, television, touch screen of a smart device, and/or the like. The graphic representation 28 includes at least one image, picture, structure, shape, illustration, indicator or the like that graphically conveys the FRP. In at least one embodiment, the graphic representation 28 includes a sparkline, which may include a chart or graph that provides a visual representation of data or a data trend in a highly condensed format. The sparkline encapsulates data in a small area and can be easily incorporated into a display near other contextual or relevant data. In an embodiment, the graphic representation 28 lacks a text message, numerical value, or audio signal. In another embodiment, the graphic representation 28 includes a numerical value 30 for the FRP, but no other text. In other embodiments, the graphic representation 28 includes a numerical value and/or a text message or audio signal related to the FRP. The graphic representation 28 of the FRP allows a clinician to intuitively understand the FRP. By viewing the graphic representation 28, the clinician is able to quickly appreciate the FRP and changes in the FRP over time, without relying solely on the numerical value 30 to determine whether fluid should be administered to the patient 14.

The FRP reporting module 24 may be operatively connected to the user interface 26, such as through a wired or wireless connection. The user interface 26 may be a keyboard, mouse, touchscreen of the display 22, voice control input device, and/or the like. The FRP reporting module 24 is configured to report the FRP upon instruction from a clinician. For example, the clinician may request reporting of the FRP by inputting a command to the FRP reporting module 24 through the user interface 26. Upon receipt of the request by the FRP reporting module 24, the FRP may be reported and shown on the display 22 until the clinician instructs the FRP reporting module 24 to cease reporting the FRP, such as through a cease reporting instruction that is received through the user interface 26. In this manner, the clinician may ensure that circumstances are appropriate for the FRP to be determined, reported, and displayed. For example, the clinician may ensure that the patient 14 is steady and still (that is, not moving) during the reporting period to reduce the risk of patient motion generating noise within the physiological signal, which may affect the FRP. Additionally, the clinician may determine if there is any patient information, such as medications, that may affect analysis of the physiological signal and/or determination of the FRP. The patient information may be input through the user interface 26, and the FRP determination module 18 may adapt, modify, or alter its analysis of the physiological signal(s) and/or determination of the FRP accordingly.

For example, once the clinician instructs the FRP reporting module 24 to report the FRP, the FRP reporting module 24 may prompt the clinician to input information regarding the patient 14, such as height, weight, body mass index (BMI), body surface area (BSA), hydration levels, skin pigmentation, whether the patient 14 is on a fluid drip, medication information, such as recent infusion of drug(s) and types of drug(s) infused, and/or the like. The FRP determination module 18 may use the patient information regarding the patient 14 to account for, adjust, or otherwise modify analysis of the physiological signal(s) and/or determination of the FRP.

In an embodiment, the monitoring device 16 may not include the FRP reporting module 24. Instead, the monitoring device 16 may include the FRP determination module 18 to determine the FRP, and the FRP display module 20 may display the graphic representation 28 of the FRP on the display 22, without receiving requests or commands from the user.

In an embodiment, the monitoring device 16 may report the FRP numerically and not graphically. In this case, the monitoring device 16 may determine the FRP through the FRP determination module 18, and use the FRP reporting module 24 to report the FRP, such as through a numerical value, on the display 22 at the instruction of the clinician, for example. As such, the monitoring device 16 may not display a graphic representation of the FRP.

The modules 18, 20, and 24 may include one or more control units, circuits, or the like, such as processing devices that may include one or more microprocessors, microcontrollers, integrated circuits, memory, such as read-only and/or random access memory, and the like. As an example, each of the modules 18, 20, and 24 may include or be formed as an integrated chip. Each of the modules 18, 20, and 24 may be separate and distinct circuits or processors within the monitoring device 16, for example. Optionally, the modules 18, 20, and 24 may be integrated into a single circuit or processor.

The modules 18, 20, and 24 may be contained within a workstation that may be or otherwise include one or more computing devices, such as standard computer hardware (for example, processors, circuitry, memory, and the like). The physiological sensor 12 may be operatively connected to the workstation, such as through a cable or wireless connection. While the physiological sensor 12 and the monitoring device 16 are shown as separate components, the physiological sensor 12 and the monitoring device 16 may be integrally part of a single unit, workstation, or the like. As an example, the modules 18, 20, and 24 may be integrally part of a blood pressure detection system, a PPG system, an echocardiography system, or the like.

Additionally, one or more of the modules 18, 20, and 24 may be housed within a smart cable, adapter, or the like, that is part of a cable assembly having one or more sensors at one end, and a connector configured to connect to a monitor at an opposite end. In this manner, the physiological sensor 12 and the modules 18, 20, and 24 may be configured to connect to a device configured to display the FRP to an individual. For example, the modules 18, 20, and 24 may be part of an assembly that connects to a device, such as a cellular or smart phone, tablet, other handheld device, laptop computer, monitor, or the like that may be configured to receive data from the assembly and show the data on a display of the device. In an embodiment, the device may be configured to download software in the form of applications configured to operate in conjunction with the assembly.

The monitoring device 16 may include any suitable computer-readable media used for data storage. For example, the modules 18, 20, and 24 may include computer-readable media. The computer-readable media are configured to store information that may be interpreted by the modules 18, 20, and 24. The information may be data or may take the form of computer-executable instructions, such as software applications, that cause a microprocessor or other such control unit within the modules 18, 20, and 24 to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

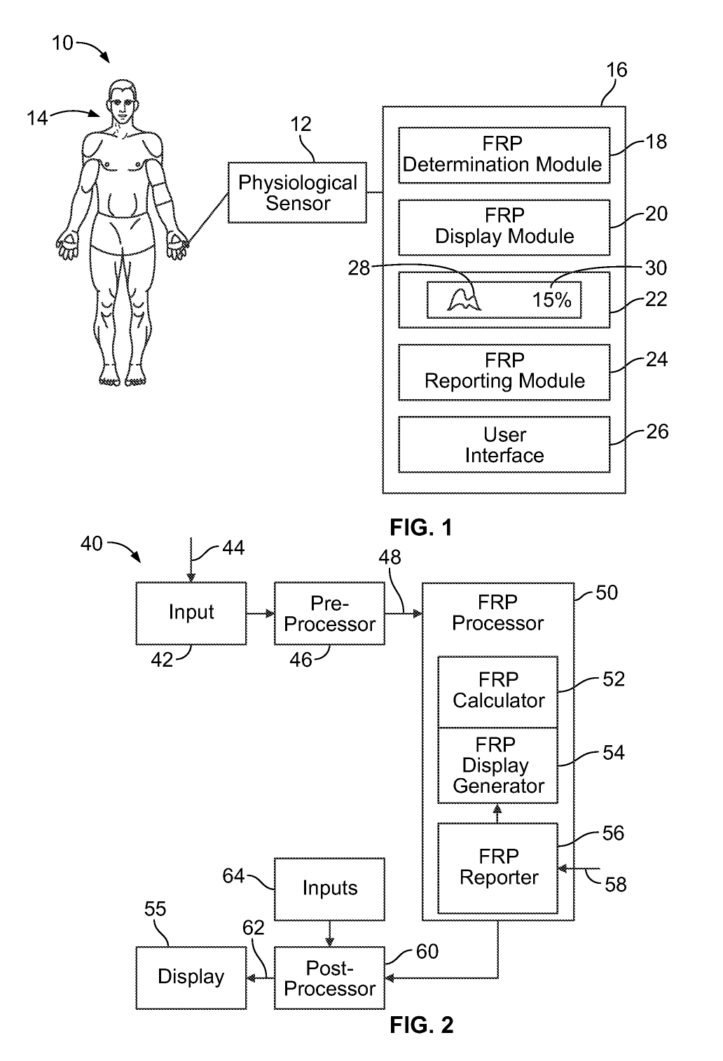

FIG. 2 illustrates a block diagram of a system 40 for displaying an FRP, according to an embodiment of the present disclosure. The system 40 includes an input 42 that receives a physiological signal 44, such as a raw blood pressure signal, PPG signal, or stroke volume signal from a sensor applied to a patient. The input 42 may include a port for wired connection to the sensor, or a wireless receiver for receiving signals wireless from the sensor. The system 40 may optionally include a pre-processor 46 that initially processes the physiological signal 44. For example, the pre-processor 46 may include one or more filters, such as a low pass filter to remove noise, and/or a filter based on the patient's heart rate to remove irregular pulses. The pre-processor 46 manipulates the incoming physiological signal 44 prior to calculation of a parameter, such as an FRP.

The pre-processed physiological signal 48 is then passed to an FRP processor 50. In at least one embodiment, the FRP processor 50 includes an FRP calculator 52, an FRP display generator 54 and an FRP reporter 56. The FRP calculator 52 takes the incoming pre-processed physiological signal 48 and calculates the FRP based on an analysis of one or more characteristics of the pre-processed physiological signal 48. The FRP display generator 54 receives the FRP from the FRP calculator 52 and generates a graphic representation of the FRP, which may be shown on a display 55 of the system 40.

The FRP reporter 56 may receive an input 58 from a user interface to instruct the FRP display generator 54 to display the FRP on the display 55. For example, a clinician may request display of the FRP as generated by the FRP display generator 54 by inputting instructions through a user interface, for example. Alternatively, the system 40 may not include the FRP reporter 56. In at least one other embodiment, the system 40 may include the FRP reporter 56, but not the FRP display generator 54. As such, the system 40 may output a numerical value for the FRP, but not a graphical representation of the FRP.

The system 40 may optionally include a post-processor 60 that further processes the FRP and/or the graphic representation of the FRP to provide smoothed or processed FRP values 62, including one or both of the numerical value and graphic representation of the FRP, prior to displaying the FRP values 62 to a clinician or caregiver. For example, the post-processor 60 may smooth the FRP values 62 by calculating a running average of the calculated FRP values over a time window. The time window may be chosen by a user for a smoother or faster FRP value (for example, 120 seconds, or 15 seconds, or other similar durations). The post-processor 60 may also remove outlier FRP values before averaging or displaying. For additional smoothing, the post-processor 60 may employ percentile averaging, in which only the middle 50% of calculated FRP values within a time window are added to the running average, and the lowest 25% and highest 25% of values are removed. Additionally, the post-processor 60 may remove particular FRP values due to other conditions that indicate a deterioration in the physiological signal 44 or the patient's condition, such as a signal-to-noise ratio value or an artifact flag (indicating a potential artifact in the physiological signal), physiological parameters being zero or out of range (for example, blood oxygen saturation or heart rate beyond a particular threshold), or other conditions (for example, arrhythmia present in the signal). The post-processor 60 may also check system settings, and may decide to remove an FRP value due to a system status, such as a gain change in a pulse oximeter, which may cause an abrupt step change in the physiological signal, leading to temporarily skewed FRP values. These various system, signal, and physiological inputs to the post-processor are labeled as inputs 64.

The system 40 also includes an output that passes the processed FRP value(s) 62 to the display 55 for displaying the FRP value(s) to the clinician or caregiver, such as a doctor or nurse or other clinician, for making clinical decisions about patient care. For example, in an embodiment, a numerical FRP value of 15% is used as a threshold for fluid therapy. If the displayed FRP value is greater than a threshold, such as 15%, then the patient is likely to benefit from fluid therapy. If the displayed FRP value is less than 15%, the patient may not benefit. Based on this determination, fluid administration may be initiated, continued, or ceased. The system 40 may provide a prompt on the display 55 when the FRP value crosses this threshold. The FRP value may be used in GDT (goal-directed therapy) to incrementally load the patient until the FRP value indicates that further fluid therapy would not be helpful. The 15% threshold is merely an example, and it is to be understood that the threshold may be greater or less than 15%. Moreover, different thresholds may be used to determine whether individual patients would benefit from fluid administration.

Either the system 10 (shown in FIG. 1) or the system 40 may be used to analyze physiological signals and display graphic representations of FRP, as described in the present application. Thus, in at least one embodiment, a system may be configured to display graphic representations of the FRP, but not report the FRP on demand at the request of an individual. Additionally, either the system 10 or the system 40 may be used to report FRPs (at the request of a clinician, for example), as described in the present application. As such, in at least one embodiment, a system may be configured to report the FRP on demand at the request of an individual and display a numerical value of the FRP, instead of a graphic representation of the FRP. At least one embodiment is used to report an FRP on demand at the request of an individual, and the reported FRP may be shown as a graphic representation on a display (as opposed to just a numerical value, for example).

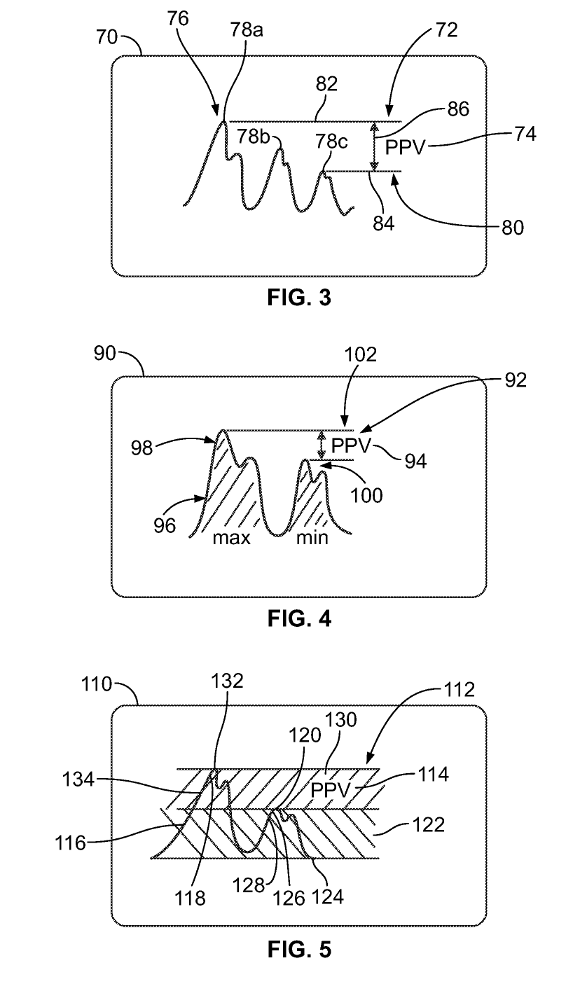

FIG. 3 illustrates a display 70 showing a graphic representation 72 of an FRP 74, according to an embodiment of the present disclosure. As shown in FIG. 3, the FRP 74 may be pulse pressure variation (PPV), which measures the variation of blood pressure pulses with respect to a maximum pulse value and a minimum pulse value over time. PPV is further described in United States Patent Publication No. 2014/0073889, entitled “Systems and Methods for Determining Fluid Responsiveness,” which is hereby incorporated by reference in its entirety. The physiological signal may be a blood pressure signal, as detected by an A-line, blood pressure cuff, and/or the like. The blood pressure signal is shown on the display as a blood pressure waveform 76 including a plurality of primary pulse peaks 78a, 78b, and 78c.

In an embodiment, the display 70 includes the PPV waveform 76 and a graphic representation 72 that indicates the FRP value. In an embodiment, the graphic representation 72 includes a difference bracket 80 graphically identifies the difference between the highest and lowest peaks 78. In FIG. 3, the difference brackets includes an upper line 82 and a lower line 84. The upper line 82 corresponds to the height of the maximum primary pulse peak 78a, as shown on the display 70, while the lower line 84 corresponds to the height of the minimum primary pulse peak 78c, as shown on the display 70. The difference bracket 80 may also include an indicator 86 of the difference between these two lines. In FIG. 3, the difference indicator 86 includes a perpendicular difference line that spans between the upper and lower lines 82 and 84 graphically shows PPV, the FRP. In other examples, the indicator 86 may be shown as a shaded area, or a different shape between the two lines 82 and 84. Alternatively, the difference bracket 80 may not include the indicator 86. Instead, the difference bracket 80 may simply include the upper and lower lines 82 and 84, respectively. Alternatively, the difference bracket 80 may include the difference indicator 86 while omitting the lines 82 and 84. Also, in various embodiments, the display 70 may omit or refrain from showing the blood pressure waveform 76, and instead simply show the graphic representation 72.

In operation, the graphic representation 72 provides a clinician with an easily recognizable visual indication of the FRP 74. The graphic representation 72, as well as the blood pressure waveform 76, may change over time. As such, the clinician may find it easier to determine whether or not to administer fluids to a patient based on the graphic representation 72, in contrast to an isolated numerical value. The display 70 may also show the numerical value of the FRP, as well.

As described above, the FRP 74 may be represented by PPV. However, the graphic representation 72 shown in FIG. 3 may be used with various other physiological signals, such as PPG signals. For example, instead of the blood pressure waveform 76, the display may show a PPG waveform having a pulse train showing multiple cardiac pulses. The graphic representation 72 may be used with respect to maximum and minimum cardiac peaks within a PPG waveform, for example. Further, while FIG. 3 shows “PPV” on the display 70, various other FRPs may be indicated on the display 70. As an example, instead of PPV, the display may show “ΔPOP” next to the graphic.

FIG. 4 illustrates a display 90 showing a graphic representation 92 of an FRP 94, according to an embodiment. The FRP 94 may be pulse pressure variation (PPV). The graphic representation 92 may include a composite image 96 that shows the maximum and minimum pulses 98 and 100, respectively, of a blood pressure signal, for example, while omitting intermediate pulses between the maximum and minimum pulses (such as peak 78b in FIG. 3). The graphic representation 92 may also include a difference bracket 102, as described above with respect to FIG. 3. Alternatively, the graphic representation 92 may not include the difference bracket 102. The displayed maximum and minimum pulses 98 and 100, respectively, of the composite image 96 may be obtained from numerous pulses taken over a time window, such as a single respiratory cycle, several respiratory cycles, or a defined time period (15 seconds, 30 seconds, etc.).

In the embodiment depicted in FIG. 4, the PPV value from a blood pressure signal is used as the FRP. However, in other embodiments, other values of the FRP may be used, such as a measure of respiratory variation from a PPG signal. The graphic representation 92 shown in FIG. 4 may also be used with these other FRP values.

FIG. 5 illustrates a display 110 showing a graphic representation 112 of an FRP 114, according to an embodiment of the present disclosure. As shown, a blood pressure waveform 116, which may represent an actual blood pressure waveform over time (as shown in FIG. 3), or a composite image showing the maximum and minimum pulses 118 and 120, respectively (as described with respect to FIG. 4) may be shown on the display 110. The graphic representation 112 includes a minimum band 122 that extends from a base 124 of the minimum pulse 120 to a top 126 of a primary peak 128 of the minimum pulse 120, and a maximum band 130 that extends from the top 126 of the primary peak 128 of the minimum pulse 120 to a top 132 of a primary peak 134 of the maximum pulse 118. Each of the minimum and maximum bands 122 and 130, respectively, may be linear bands that extend across the display 110, as shown. The minimum band 122 may be shaded a first shade or texture, or colored a first color, while the maximum band 130 may be shaded a second shade or texture (that differs from the first), or colored a second color (that differs from the first). Accordingly, a clinician is able to easily and intuitively determine whether or not to administer fluid to the patient by the size (e.g., the height) of the maximum band 130 with respect to the minimum band 122. That is, the FRP 114, in the form of PPV, is represented by the height (from the top 126 of the primary peak 128 of the minimum pulse 120 to the top 132 of the primary peak 134 of the maximum pulse 118) or area of the maximum band 130.

Alternatively, the graphic representation 112 may not include the minimum band 122. Instead, the graphic representation 112 may include just the maximum band 130.

As shown in FIG. 5, the FRP 114 may be represented by PPV. However, the graphic representation 112 may be used with various other physiological signals, such as PPG signals. For example, the maximum and minimum bands 122 and 130 shown in FIG. 5 may be used with respect to a PPG signal, for example.

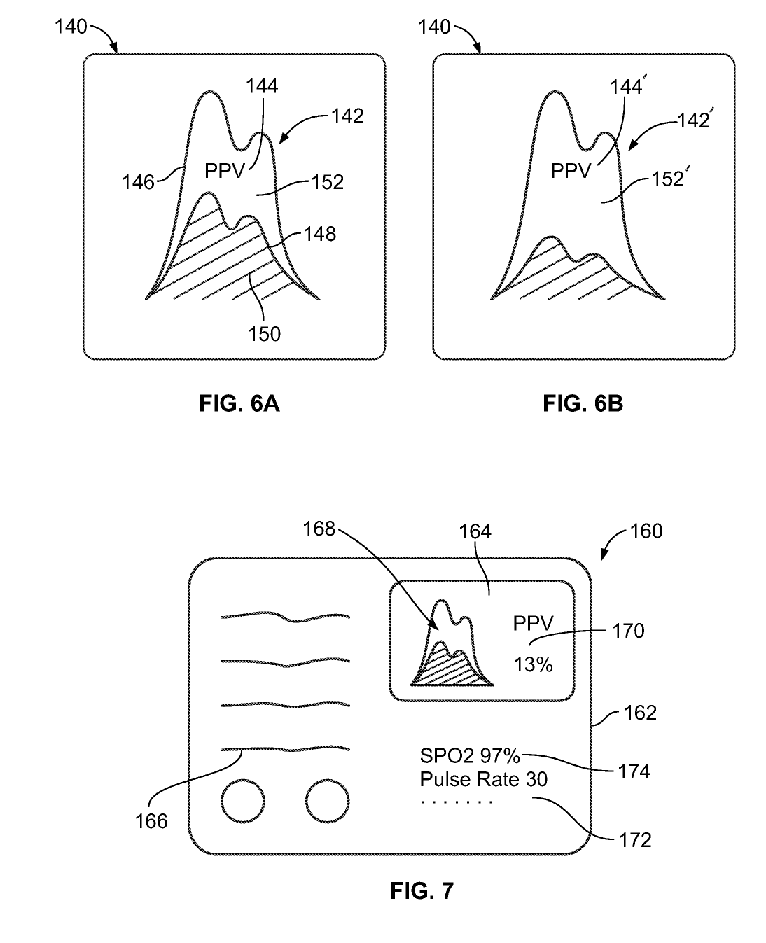

FIG. 6A illustrates a display 140 showing a graphic representation 142 of an FRP 144, according to an embodiment of the present disclosure. In this embodiment, the graphic representation 142 may include a minimum pulse 148 superimposed on or overlapping a maximum pulse 146, or vice versa. The FRP 144, in the form of PPV or other forms of FRP, is indicated by the difference between the maximum pulse 146 and the minimum pulse 148. The area 150 under the curve of the minimum pulse 148 may be shaded, textured, or colored, while the area 152 between the curve of the maximum pulse 146 and the curve of the minimum pulse 148 may be shaded, textured, or differently, to visually distinguish the two pulses. Alternatively, one area may be shaded, colored, or textured, and the lines 146 and 148 may simply indicate the respective areas. The FRP 144 may be easily and intuitively identified by the area 152 between the two pulses.

The graphic representation 142 provides a compact visual indication showing both the maximum and minimum pulses 146 and 148 together, and may be used with respect to displays having limited surface area. That is, the compact graphic representation 142 may fit onto a small screen, for example.

Alternatively, a mean pulse wave (obtained, for example, by ensemble averaging the pulses of the physiological signal over a number of cycles) may also be shown on the graphic representation 142. The mean pulse wave may be shown for reference in relation to the maximum and minimum pulses 146 and 148. The mean pulse wave may be shown as a dashed line, a colored line that differs from the maximum and minimum pulses 146 and 148, and/or the like.

Alternatively, the area 150 may not be shown, shaded or colored. Instead, because the area 152 shows the FRP, only the area 152 may be shown.

As shown, the FRP 144 may be represented by PPV. However, the graphic representation 142 may be used with various other physiological signals, such as PPG signals. For example, the area between maximum and minimum pulses of a PPG signal may represent the FRP and be shown on the display.

FIG. 6B illustrates the display 140 showing the graphic representation 142′ of the fluid responsiveness predictor 144′, according to an embodiment of the present disclosure. As shown in FIG. 6B, the graphic representation 142′ may change over time. Notably, in FIG. 6B, the area 152′ has grown in relation to the area 152 shown in FIG. 6A. As such, the graphic representation 142′ is dynamic. All of the graphic representations shown and described, including the various areas, pulses, and shapes, may change over time and, as such, may dynamically show the FRP.

FIG. 7 illustrates a front view of a monitoring device 160, according to an embodiment of the present disclosure. The monitoring device 160 may include a housing 162 that houses the modules 18, 20, 24, as shown in described with respect to FIG. 1, or the pre-processor 46, FRP processor 50, and post processor 60, as shown and described with respect to FIG. 2. The monitoring device 160 may also include a display 164 and a user interface 166, such as any of those described above. The display 164 may show a graphic representation 168 of an FRP, such as any of those described above, as well as a numerical value 170 of the FRP. The monitoring device 160 may also show various other physiological characteristics, such as pulse rate 172, blood oxygen saturation 174, and/or the like.

As described above, the FRP may represent a PPV of a blood pressure signal, for example. Alternatively, the FRP may represent a respiratory modulation of a PPG signal, as described below.

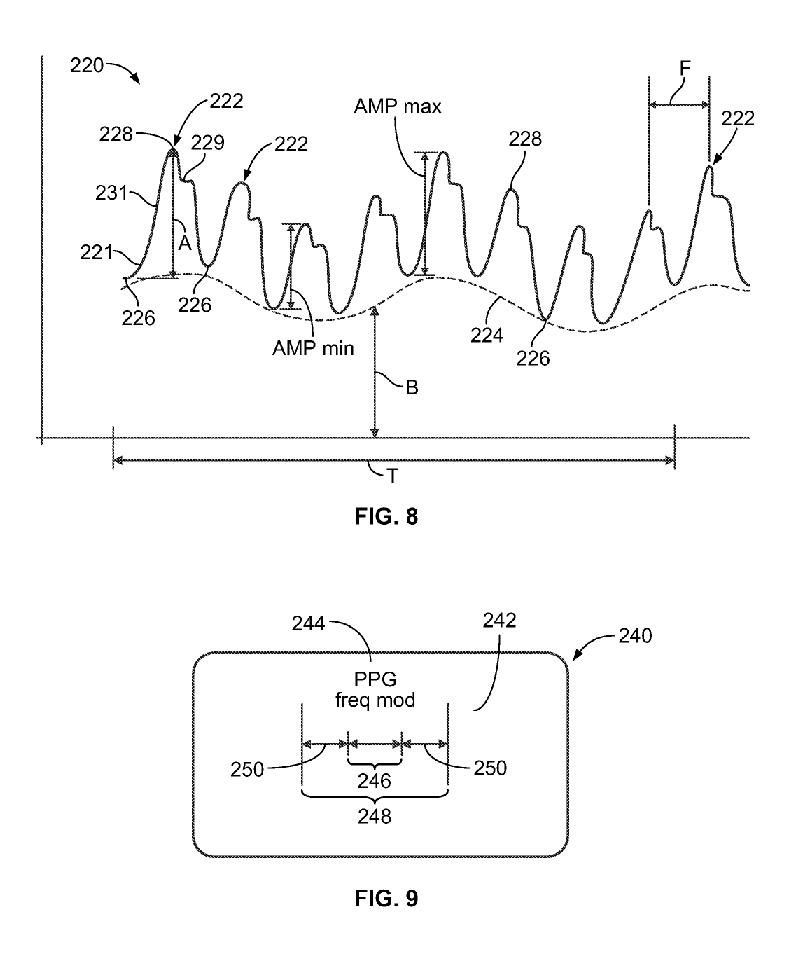

FIG. 8 illustrates a representation of a PPG signal 220, according to an embodiment of the present disclosure. The PPG signal 220 may be obtained from a pulse oximeter, for example and is an example of a physiological signal, such as described above. The PPG signal 220 may be output as a PPG waveform 221 that represents the absorption of light by a patient's tissue over time. The PPG waveform 221 includes cardiac pulses 222, where absorption of light increases due to the increased volume of blood in the arterial blood vessel due to the cardiac pulse 222. Each cardiac pulse 222 may be identified based on a valley 226, peak 228, dichrotic notch 229, and subsequent valley 226. The PPG signal includes an upstroke 231 with an amplitude A, measured from the preceding valley 226 to the peak 228. Other amplitude values may be derived from the PPG waveform, such as downstroke amplitude, average amplitude, or area under the pulse 222. The PPG waveform 221 also includes a baseline shift B indicating a baseline level 224 of the light absorption. The PPG waveform 221 modulates above the baseline level 224 due to the arterial blood pulses.

For at least some patients, the PPG signal 220 is affected by the patient's respiration—inhaling and exhaling. A segment of a PPG waveform 221 during normal breathing is shown in FIG. 8. The waveform 221 includes the cardiac pulses 222. It should be noted that the number of cardiac pulses 222 per breath is not necessarily to scale, and may vary from patient to patient. Respiration (breathing in and out) may cause modulations in the PPG waveform 221.

One respiratory modulation is a modulation of the baseline B of the PPG waveform 221. The effect of the patient's breathing in and out causes the baseline 224 of the waveform 221 to move up and down, cyclically, with the patient's respiration rate. The baseline 224 may be tracked by following any component of the PPG waveform 221, such as the peaks 228, valleys 226, dichrotic notches 229, median value, or other value. A second respiration-induced modulation of the PPG signal 220 is a modulation of the amplitude A. As the patient breathes in and out, the amplitude A of each cardiac pulse 222 decreases and increases, with larger amplitudes tending to coincide with the top of the baseline shift B, and smaller amplitudes tending to coincide with the bottom of the baseline shift B (though the larger and smaller amplitudes do not necessarily fall at the top and bottom of the baseline shift). A third respiratory modulation is modulation of the frequency F between cardiac pulses. Each of these three modulations may be referred to as a respiratory component of the PPG signal 220, or a respiratory-induced modulation of the PPG signal 220. It should be noted that a particular individual may exhibit only the baseline modulation, or only the amplitude modulation, or both. As referred to herein, a respiratory component of the PPG signal 220 includes any one of these respiratory-induced modulations of the PPG waveform 221, a measure of these modulations, or a combination of them.

The respiratory modulations of the PPG waveform 221 can be affected by a patient's fluid responsiveness. For example, a patient that is fluid responsive (for example, a hypovolemic patient) may exhibit relatively larger respiratory variations of the PPG waveform 221, while a patient that is not fluid responsive may exhibit relatively smaller respiratory variations of the PPG waveform 221. When a patient loses fluid, the respiratory variations present in the patient's PPG signal 220 tend to increase. As an example, when the patient's fluid volume is low, the arterial system exhibits larger compliance and thus expands more with each cardiac pulse, relative to the baseline 224. Both the baseline modulation and the amplitude modulation may become more pronounced when a patient's fluid volume decreases. Thus, larger respiratory modulations may indicate that a patient is in need of fluids, while smaller respiratory modulations may indicate that a patient is not in need of fluids. The respiratory modulations of the PPG signal 220, such as the PPG waveform 221, may be identified and used to predict a patient's fluid responsiveness. Accordingly, each respiratory modulation of the PPG signal 220 may provide an FRP.

In an embodiment, a medical monitoring system, such as the systems 10 and 40 described above, receives a PPG signal and calculates an FRP based on the PPG signal. In at least one embodiment, the FRP is a measure of a patient's likelihood of response to fluid therapy. As an example, the FRP represents a prediction of whether such fluid therapy will improve blood flow within the patient. In at least one embodiment, the FRP is a metric that reflects a degree of respiratory variation of the PPG signal. One example of an FRP metric is a measure of the amplitude modulations of the PPG signal, such as ΔPOP, as described below. In other embodiments, the FRP metric is a measure of the respiratory variation of the PPG, such as a measure of the baseline modulation of the PPG, or other suitable metrics assessing the respiratory modulation of the PPG. For example, an FRP may be based on the amplitudes or areas of acceptable cardiac pulses 222 within a particular time frame or window. The minimum amplitude of the cardiac pulses 222 may be subtracted from the maximum amplitude then divided by an average or mean value. Alternatively, an FRP may be derived from a frequency of cardiac pulses 222 within a time frame or window. For example, a modulation or variation in frequency among two or more cardiac pulses 222 may be used to derive an FRP. In general, the FRP may be based on one or more respiratory variations exhibited by the PPG signal 220. Further, an FRP may be determined through the use of wavelet transforms, such as described in United States Patent Application Publication No. 2010/0324827, entitled “Fluid Responsiveness Measure,” which is hereby incorporated by reference in its entirety.

In at least one embodiment, ΔPOP is used as the FRP. The ΔPOP metric is calculated from the PPG waveform 221 for a particular time window as follows:

ΔPOP=(AMPₘₐₓ−AMPₘᵢₙ)/AMPₐᵥₑ (1)

where AMPₘₐₓ represents the maximum upstroke amplitude (amplitude from a pulse minimum to a pulse maximum) during the time window (such as time window T in FIG. 1), AMPₘᵢₙ represents the minimum upstroke amplitude during the time window, and AMPₐᵥₑ is the average of the two, as follows:

AMPₐᵥₑ=(AMPₘₐₓ+AMPₘᵢₙ)/2 (2)

In other embodiments, AMPₘₐₓ and AMPₘᵢₙ may be measured at other locations of the PPG signal, such as within or along a pulse. ΔPOP is a measure of the respiratory variation in the AC portion of the PPG signal. ΔPOP is a unit-less value, and can be expressed as a percentage. In at least one embodiment, the time window is one respiratory cycle (inhalation and exhalation). In at least one other embodiment, the time window is a fixed duration of time that approximates one respiratory cycle, such as 5 seconds, 10 seconds, or another duration. In other embodiments, the time window may be adjusted dynamically based on the patient's calculated or measured respiration rate, so that the time window is approximately the same as one respiratory cycle. A signal turning point detector may be used to identify the maximum and minimum points in the PPG signal, in order to calculate the upstroke amplitudes. In some embodiments, AMPₘₐₓ and AMPₘ₁ₙ may be calculated by identifying a maximum value and a minimum value within a cardiac pulse window, and calculating a difference between those values. This difference may correspond with an upstroke or a downstroke, for example.

Referring to FIGS. 1 and 8, for example, the physiological sensor 12 may be a pulse oximetry sensor that detects the physiological signal as a PPG signal of the patient 14. The FRP determination module 18 analyzes the PPG signal and determines an FRP based on at least one characteristic of the PPG signal. For example, the FRP may be ΔPOP of the PPG signal. Alternatively, the FRP may be a PPG variation, as determined from a maximum pulse in relation to a minimum pulse, similar to as described above with respect to PPV. Also, alternatively, the FRP may be based on the baseline 224, such as through comparison of a minimum baseline peak and a maximum baseline peak, similar to as described above with respect to PPV. Also, alternatively, the FRP may be a maximum frequency between neighboring cardiac pulses 222 and a minimum frequency between neighboring cardiac pulses 222.

The FRP display module 20 receives the FRP from the FRP determination module 18 and generates a graphic display of the FRP. When the FRP is ΔPOP, for example, the FRP display module 20 may generate a graphic representation with respect to the maximum and minimum cardiac pulse signals, similar to as described above with respect to FIGS. 3-7. Similar, when the FRP is in relation to maximum and minimum peaks of the baseline 224, the FRP display module 20 may generate a graphic representation with respect to the baseline peaks similar to as described above with respect to FIGS. 3-7.

FIG. 9 illustrates a display 240 showing a graphic representation 242 of an FRP 244, according to an embodiment. The FRP 244 may be a frequency modulation of a PPG signal. The graphic representation 242 may include a minimum frequency representation 246 between first and second neighboring cardiac pulses of a PPG waveform, and a maximum frequency representation 248 between third and fourth neighboring cardiac pulses of the PPG waveform. The FRP 244 may be shown as the difference 250 between the minimum and maximum frequency representations 246 and 248. As shown in FIG. 9, the graphic representation 242 may be a linear bracket. Alternatively, the graphic representation 242 may be or include color coded bands, circles, or other shapes. For example, a shape showing the difference between the minimum and maximum frequencies may be shown as a first color, while a shape showing the minimum frequency may be a second color that is different from the first color. Also, alternatively, the graphic representation 242 may be based on minimum and maximum frequencies of peaks of the baseline 224, as opposed to cardiac pulses.

As described above, the FRP may be based on a PPV of a blood pressure signal, or a respiratory modulation of a PPG signal, or a difference between a stroke volume variation (SW) between a maximum stroke volume and a minimum stroke volume, as described below.

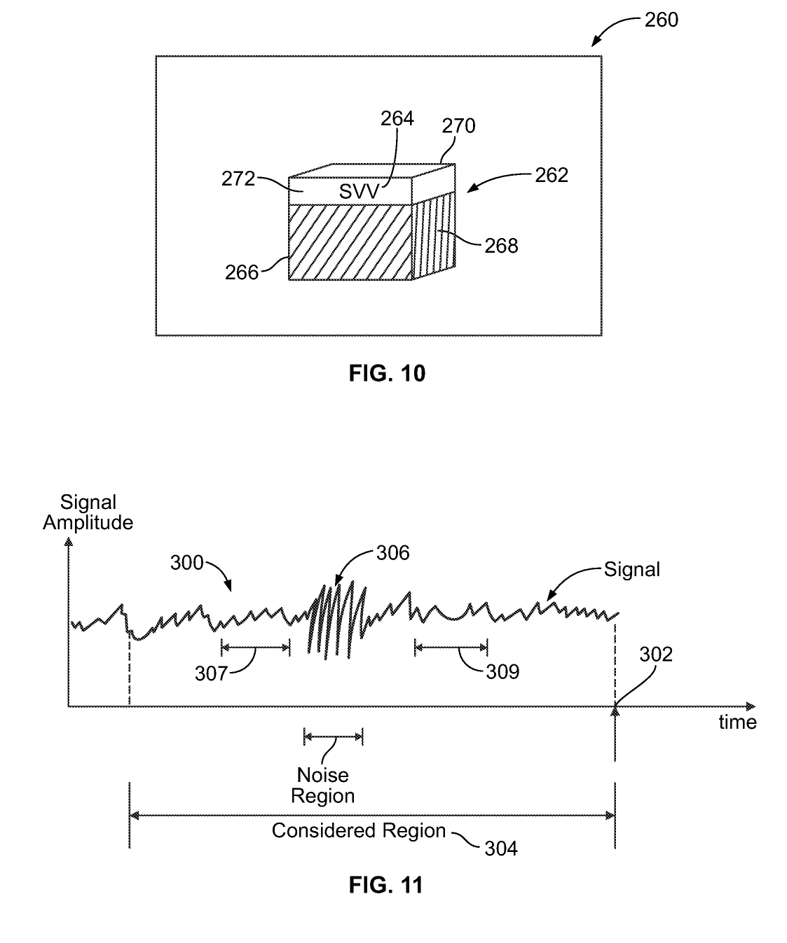

FIG. 10 illustrates a display 260 showing a graphic representation 262 of an FRP 264, according to an embodiment. Referring to FIGS. 1 and 10, the physiological signal may be stroke volume, as detected by the physiological sensor 12, such as an echocardiographic device. The FRP determination module 18 receives the stroke volume signal from the physiological sensor 12 and determines the FRP as a stroke volume variation (SW), which may be a difference between a maximum stroke volume and a minimum stroke volume, for example, within one or more cardiac cycles. The FRP display module 20 may generate the graphic representation 262 based on the determined SW. The graphic representation 262 may be a shape, such as a cube 266 that indicates a volume that represents a minimum stroke volume 268 and a maximum stroke volume 270. The minimum stroke volume 268 is within the maximum stroke volume 270. The depicted difference 272 between the maximum stroke volume 270 and the minimum stroke volume 268 is indicative or representative of the FRP 264. The minimum stroke volume 268 may be shaded or colored as a first shade or color, while the difference 272 may be shaded or colored as a second shade or color. Alternatively, the display 260 may show only the depicted difference 272.

While the shape is described as a cube 268, various other shapes and sizes may be used. For example, instead of a representation of a three dimensional object, the shape may be a square, rectangle, triangle, circle, heart-shape, or the like. Similarly, instead of the cube 268, a sphere, pyramid, or the like may be used.

Referring to FIGS. 1-10, the monitoring device 16 (shown in FIG. 1) or the FRP processor 50 may be used to determine an FRP based on received physiological signals (such as blood pressure signals, PPG signals, stroke volume signals, and/or the like) and generate a graphic representation of the FRP, such as any of those described above. Additionally, numerical values and text may be displayed providing additional information regarding the FRP. Further, the systems may generate audio signals in conjunction with the FRPs shown. For example, if an FRP passes a threshold for fluid loading, the graphic representation of the FRP may change color, flash, or the like, a text message may be displayed indicated “Administer Fluid,” and/or an audio signal, such as a buzzing, beeping, or recorded voice indicating “Administer Fluid,” may be output.

Moreover, any of the graphic representations described above may be dynamic. For example, maximum pulses may expand and recede with respect to minimum pulses over a respiratory cycle. Further, as shown in FIG. 10, the maximum stroke volume may expand outwardly away, and recede inwardly toward the minimum stroke volume during one or more cardiac cycles. The FRP can be clearly and quickly communicated via the dynamically changing graphical representation. The graphical representation changes with the changing FRP, but remains a simple visual tool that can convey this changing information quickly without complicated historical trends or complex figures. For example, the display 140 shown in FIGS. 6A and 6B shows a graphical representation that changes over time. Further, the graphical representations described in the present application may or may not represent real time FRPs. Instead, the graphical representations may represent moving averages, and/or may be periodically updated (such as every second or minute).

As described above, the FRP may be continually determined and displayed while the patient is being monitored. In at least one embodiment, the FRP reporting module 24 (shown in FIG. 1) or the FRP reporter 56 (shown in FIG. 2) may be used to report the FRP, numerically and/or graphically (as described above) based on input received from a user interface.

Either of the FRP reporting module 24 or the FRP reporter 56 may be used to report an FRP on demand. In at least one embodiment, the clinician requests the FRP from the system by, for example engaging a user interface, such as by pushing a button, or clicking a field on the screen. Once the FRP reporting module 24 receives the reporting request, the FRP determination module 18 may be determined and reported from the requested time.

FIG. 11 illustrates a physiological signal 300 over time, according to an embodiment of the present disclosure. As shown, the physiological signal 300 may be detected before a requested time 302. Referring to FIGS. 1 and 11, for example, the physiological signal 300 may be analyzed by the FRP determination module 18 to determine a dynamic FRP even before the requested time 302. However, the FRP determination module 18 may not be reported, such as through a numerical value or graphic representation, until a time after the user enters a reporting request through the user interface 26.

Once the user enters the reporting request at the requested time 302, the FRP reporting module 24, for example, may consider a time period that the physiological signal 300 is detected (for example, several minutes, or several tens of minutes) in order to calculate a reported FRP. For example, after the reporting request is entered, the FRP reporting module 24 may begin to analyze the signal from a time before the requested time 302. The considered period of time of the physiological signal 300 is shown as the considered region 304. As such, the FRP reporting module 24 may analyze representative segments of the physiological signal 300 to be employed by the FRP determination module 18 to determine the FRP.

For example, as shown, the physiological signal 300 may include noise segments 306, which may be generated by patient movement, for example. The FRP reporting module 24 may ignore the noise segments 306 and/or instruct the FRP determination module 18 to refrain from analyzing the noise segments 306 in determining the FRP. For example, the noise segments 306 may exceed a predefined threshold for an acceptable physiological signal, and may thus be discarded as noise segments 306. Thus, the FRP is calculated based on remaining regions of the signal 300 with the noise segments 306 removed. Additionally, or alternatively, once the FRP reporting module 24 instructs the FRP determination module 18 to determine the FRP over the considered region or time period, the FRP reporting module 24 may provide an average or mean value of the FRP over the considered region 304, which may then be reported as a numerical value and/or a graphic representation, as described above. The averaging of the FRP over the considered region may smooth out any aberrations in the physiological signal, such as caused by patient movement, and the like.

Once an FRP report is requested by an individual, the FRP may be reported until the monitor receives a deactivation request from the user, such as by inputting a cease reporting instruction through the user interface. Alternatively, the FRP reporting period may last from the requested time 302 until a defined end time, such as 30 seconds from the requested time. However, the predefined end time may be shorter or longer than 30 seconds, and may be user adjustable.

Additionally, before the FRP reporting module 24 initiates the requested time for reporting the FRP, the FRP reporting module 24 may prompt the clinician to input patient information. The FRP reporting module 24 may prevent the FRP from being reported until after all the requested information is input. For example, the FRP reporting module 24 may display various questions on the display regarding patient information, such as height, weight, BMI, BSA, hydration levels, pigmentation, whether the patient is on a drip, recent infusion of a medication/drug, the type of medication/drug infused, and/or the like. Once the clinician inputs the information, the FRP determination module 18 may adjust analysis of the physiological signal and/or determination of the FRP based on the input information. Alternatively, the FRP reporting module 24 may initiate reporting of the FRP without requesting patient information.

For example, the patient's skin pigmentation may be input in order to adjust analysis of the physiological signal. As an example, skin pigmentation may affect a PPG signal. As such, the output of a PPG signal for a patient of one type of skin pigmentation may differ from that of another patient having a different skin pigmentation. The FRP reporting module 24 may instruct the FRP determination module 18 to adjust analysis of the physiological signal, such as a PPG signal, accordingly. The FRP determination module 18 may have data stored in memory to adjust analysis of the physiological signal and/or determination of the FRP based on skin pigmentation, for example. As an example, signal amplitude measurements may be adjusted, such as by being increased, to compensate for increased light absorbance in patients with darker pigmentations.

Similarly, the PPG signal may be affected by medication within the circulatory system of the patient. For example, a vasopresser may affect a PPG signal. Accordingly, the output of a PPG signal for a patient on a particular medication may differ from that of another patient that is not on medication, or a different type of medication. The FRP reporting module 24 may instruct the FRP determination module 18 to adjust analysis of the physiological signal, such as a PPG signal, accordingly. The FRP determination module 18 may have data stored in memory to adjust analysis of the physiological signal and/or determination of the FRP based on medication/drugs within a circulatory system of a patient. As an example, vasoconstriction or dilation caused by drug administration may alter the amplitude modulations and may be accounted for and corrected.

The ability to inform the FRP determination module 18 that a drug with particular effects on the physiological signal, such as a PPG signal, may allow the FRP determination module 18 to account for the drug effects on the physiological signal, the FRP, and/or the threshold against which the FRP is compared in order to determine whether a patient should be given fluids.

The FRP reporting module 24 shown in FIG. 1 (or the FRP reporter 56 shown in FIG. 2) may be used in relation to any of the physiological signals and FRPs described above. By waiting to report an FRP based on a user input (e.g., “on-demand”), a clinician may ensure that the circumstances are appropriate for FRP determination, such as by limiting patient movement, for example, or removing other noise sources, to reduce motion artifacts or other interference in the physiological signal so as to provide an accurate FRP. As noted, the FRP determination module 18 may continually determine an FRP, even if no user input is received. However, the FRP reporting module 24 may ensure that an FRP is reported (for example, shown on a display as a numeric value and/or a graphic representation) when the clinician affirmatively inputs a request for an FRP into the FRP reporting module 24 through the user interface 26.

Through the use of the FRP reporting module 24, the reported FRP may be a best estimate of the FRP that may exclude noise data, for example. The FRP may be determined over a time window, such as a 5 minute, 10 minute, or longer time window that spans from an initial time period (such as the beginning of the considered region 304) to at least the requested time 302. The FRP determination module 18 may ignore physiological signal data that is above or below a predetermined threshold during the time window in order to remove high and low values when determining the FRP.

Additionally or alternatively, the FRP determination module 18 may determine several FRPs from separate sub-segments 307 and 309, for example, of the physiological signal 300 over the considered region 304. The FRPs may be used to generate a more accurate estimate of the true value of the FRP by, for example, removing outlying values, polling the values, weighting the values according to signal quality and or the additional user input, and/or the like.

Additionally, the FRP reporting module 24 may prevent the FRP from being displayed if the signal quality of the physiological signal is determined to be poor or low enough that no useful value is able to be calculated.

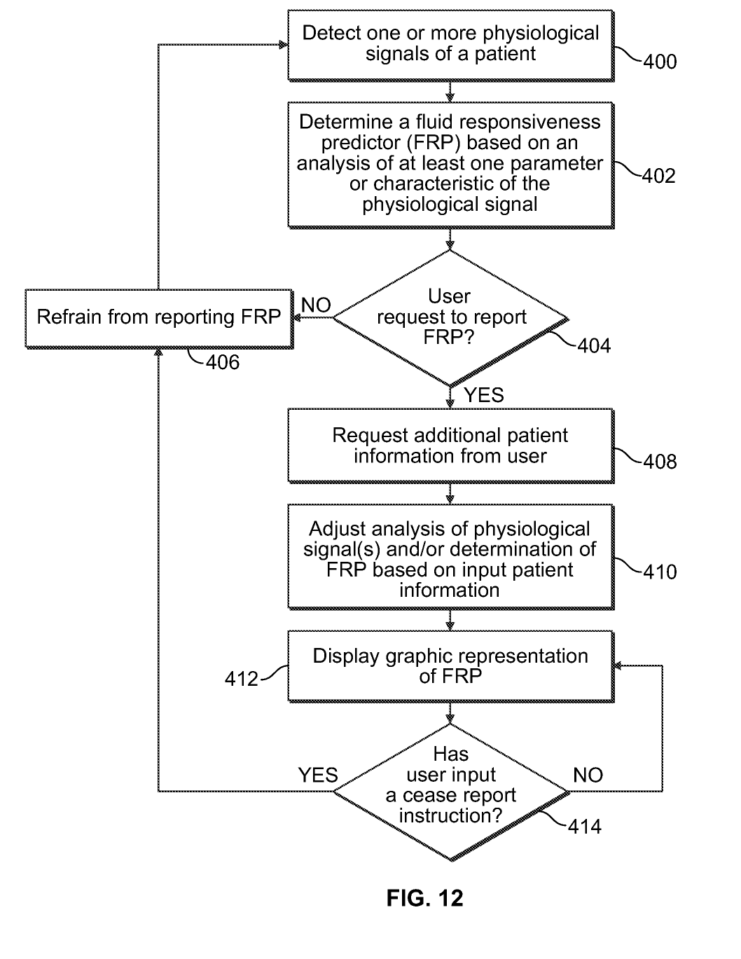

FIG. 12 illustrates a flow chart of a process of displaying an FRP, according to an embodiment of the present disclosure. The process begins at 400, in which one or more physiological signals of a patient are detected. For example, a physiological sensor may be used to detect the physiological signal(s), such as a blood pressure signal, a PPG signal, a stroke volume signal, or the like.

After detection of the physiological signal(s), an FRP is determined at 402 based on analysis of at least one parameter or characteristic of the physiological signal(s). For example, a blood pressure or a PPG signal may be analyzed to determine a maximum pulse and a minimum pulse. The FRP may be the difference between the maximum pulse and the minimum pulse.

At 404, it is determined whether a user has requested that the FRP be reported. For example, the user may input a report request through a user interface in communication with an FRP reporting module or reporter. If no report request has been entered, the system refrains from reporting the FRP at 406, and the process returns to 400.

If, however, a report request has been entered, the process continues to 408, in which the user may be requested to input patient information about the patient, such as height, weight, BMI, hydration level, skin pigmentation, presence of medication with the patient's body, and/or the like. At 410, based on the input patient information, the analysis of the physiological signals and/or determination of the FRP may be adjusted.

The process then continues to 412, in which a graphic representation of the FRP is displayed. A numerical value of the FRP may also be displayed. Alternatively, the process may proceed directly from 402 to 412.

After the graphic representation of the FRP is displayed at 412, the process may continue to 414, in which it is determined whether a user has input a cease report instruction. If the user has input a cease report instruction, the process continues to 406, and then 400. If, however, the user has not input a cease report instruction, the process may loop back to 412.

As described above, embodiments of the present disclosure provide systems and methods for displaying a graphical representation of an FRP, which may be more intuitive and easier to understand than an isolated numerical value of an FRP. Also, embodiments of the present disclosure provide systems and methods for reporting an FRP on demand, such as through a report request input through a user interface.

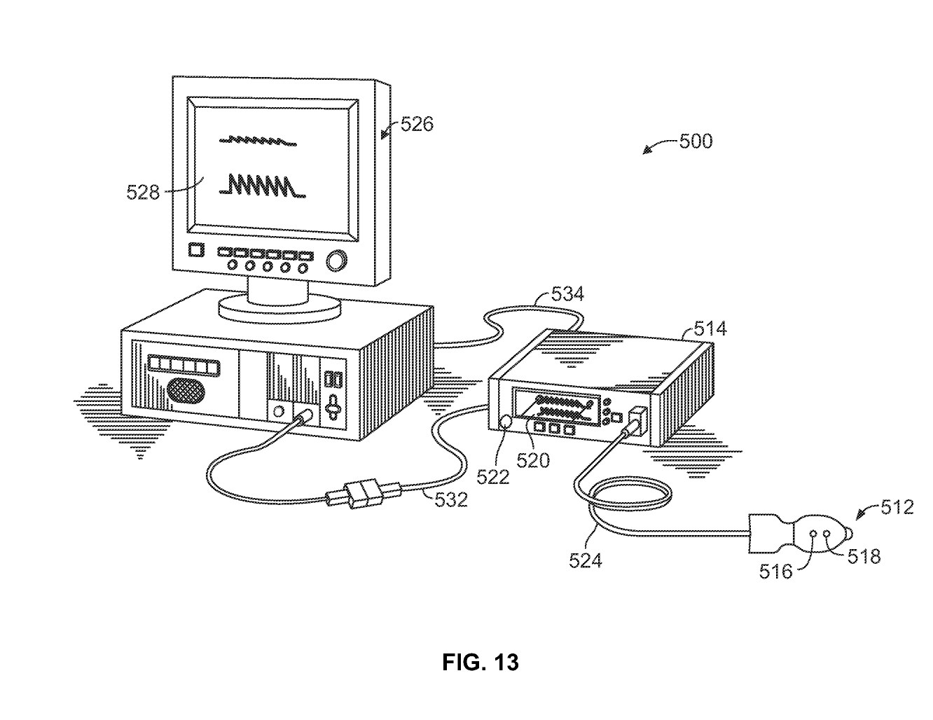

FIG. 13 illustrates a perspective view of a monitoring system 500, according to an embodiment of the present disclosure. The system 500 may be an example of, or include, a physiological sensor, such as a PPG sensor. The system 500 may include a sensor unit 512 and a monitor 514. In at least one embodiment, the sensor unit 512 may be part of a continuous, non-invasive blood pressure (CNIBP) monitoring system and/or an oximeter. The sensor unit 512 may include an emitter 516 for emitting light at one or more wavelengths into an individual's tissue. A detector 518 may also be provided in the sensor unit 512 for detecting the light originally from emitter 516 that emanates from patient tissue after passing through the tissue. Any suitable physical configuration of the emitter 516 and the detector 518 may be used. In at least one embodiment, the sensor unit 512 may include multiple emitters and/or detectors, which may be spaced apart.

According to at least one embodiment, the emitter 516 and the detector 518 may be on opposite sides of a digit such as a finger or toe, in which case the light that is emanating from the tissue has passed completely through the digit. In an embodiment, the emitter 516 and the detector 518 may be arranged so that light from the emitter 516 penetrates the tissue and is reflected by the tissue into the detector 518, such as in a sensor designed to obtain pulse oximetry data from an individual's forehead.

In at least one embodiment, the sensor unit 512 may be connected to and draw its power from the monitor 514, as shown. In at least one other embodiment, the sensor unit 512 may be wirelessly connected to the monitor 514 and include its own battery or similar power supply (not shown). The monitor 514 may be configured to calculate physiological characteristics or parameters (e.g., pulse rate, blood pressure, blood oxygen saturation) based at least in part on data relating to light emission and detection received from the sensor unit 512. Further, the monitor 514 may include a display 520 configured to display the physiological parameters or other information about the system 500. The monitor 514 may also include a speaker 522.

In an embodiment, the sensor unit 512 may be communicatively coupled to the monitor 514 via a cable 524. However, in other embodiments, a wireless transmission device (not shown) or the like may be used instead of or in addition to cable 524.

The system 500 may include a multi-parameter patient monitor 526. The monitor 526 may include a cathode ray tube display, a flat panel display (as shown) such as a liquid crystal display (LCD) or a plasma display, or may include any other type of monitor now known or later developed. The multi-parameter patient monitor 526 may be configured to calculate physiological parameters and to provide a display 528 for information from the monitor 514 and from other medical monitoring devices or systems (not shown). For example, the multi-parameter patient monitor 526 may be configured to display an estimate of an individual's blood oxygen saturation generated by the monitor 514 (referred to as a “SpO₂” measurement), pulse rate information from the monitor 514 and blood pressure from the monitor 514 on the display 528.

The monitor 514 may be communicatively coupled to the multi-parameter patient monitor 526 via a cable 532 or 534 that is coupled to a sensor input port or a digital communications port, respectively and/or may communicate wirelessly (not shown). In addition, the monitor 514 and/or the multi-parameter patient monitor 526 may be coupled to a network to enable the sharing of information with servers or other workstations (not shown). The monitor 514 may be powered by a battery (not shown) or by a conventional power source such as a wall outlet.

Pulse oximeters, in addition to providing other information, can be utilized for continuous non-invasive blood pressure monitoring. For example, PPG and other pulse signals obtained from multiple probes can be processed to calculate the blood pressure of an individual. In particular, blood pressure measurements may be derived based on a comparison of time differences between certain components of the pulse signals detected at each of the respective probes. As described in U.S. Patent Application Publication No. 2009/0326386, entitled “Systems and Methods For Non-Invasive Blood Pressure Monitoring,” the entirety of which is incorporated herein by reference, blood pressure can also be derived by processing time delays detected within a single PPG or pulse signal obtained from a single pulse oximeter probe. In addition, as described in U.S. Pat. No. 8,398,556, entitled “Systems and Methods For Non-Invasive Continuous Blood Pressure Determination,” the entirety of which is incorporated herein by reference, blood pressure may also be obtained by calculating the area under certain portions of a pulse signal. Also, as described in U.S. Patent Application Publication No. 2010/0081945, entitled “Systems and Methods for Maintaining Blood Pressure Monitor Calibration,” the entirety of which is incorporated herein by reference, a blood pressure monitoring device may be recalibrated in response to arterial compliance changes.

The system 500 is shown as one example of a system configured to detect physiological signals, such as PPG signals. However, embodiments of the present disclosure may be used with various other systems configured to detect various other physiological signals, such as blood pressure signals and stroke volume signals.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “module.”

The computer or processor executes a set of instructions that are stored in one or more storage elements, in order to process data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

The block diagrams of embodiments herein may illustrate one or more modules. It is to be understood that the modules represent circuit modules that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hardwired to perform the functions described herein. Optionally, the hardware may include electronic circuits that include and/or are connected to one or more logic-based devices, such as microprocessors, processors, controllers, or the like. Optionally, the modules may represent processing circuitry such as one or more field programmable gate array (FPGA), application specific integrated circuit (ASIC), or microprocessor. The circuit modules in various embodiments may be configured to execute one or more algorithms to perform functions described herein. The one or more algorithms may include aspects of embodiments disclosed herein, whether or not expressly identified in a flowchart or a method.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, lower, mid, lateral, horizontal, vertical, front, and the like may be used to describe embodiments of the present disclosure, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” may be used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112(f) unless and until such claim limitations expressly use the phrase “means for” followed by a statement of function void of further structure.

What is claimed is:

1. A method for displaying a fluid responsiveness predictor (FRP) based on an analysis of one or more physiological signals, the method comprising:
receiving a physiological signal of the patient;
determining an FRP, including analyzing at least one characteristic of the physiological signal over time to determine the FRP;
receiving through a user interface a request to report the FRP at a requested time;
generating a reported FRP; and
displaying the reported FRP on a display until a cease report input is received through the user interface or until a defined end time for a reported FRP, wherein displaying comprises displaying the FRP using at least one graphic representation.

2. The method of claim 1, further comprising refraining from displaying the FRP in the absence of a received user request.

3. The method of claim 1, wherein the reported FRP is based on a considered time period that extends from an initial time to at least the requested time.

4. The method of claim 1, wherein generating the reported FRP comprises discarding a noisy portion of the physiological signal.

5. The method of claim 1, wherein generating the reported FRP comprises generating an average of the FRP over a considered time period that extends from an initial time to at least the requested time.

6. The method of claim 1, wherein the at least one graphic representation comprises a difference bracket.

7. The method of claim 6, wherein the difference bracket includes:
an upper line extending from a maximum peak value of a portion of the physiological signal;
a lower line extending from a minimum peak value of the portion of the physiological signal; and
a difference indicator extending between the upper line and the lower line.

8. The method of claim 1, wherein the at least one graphic representation comprises a shaded or colored area between a maximum peak value of a portion of the physiological signal and a minimum peak value of the portion of the physiological signal.

9. The method of claim 1, wherein the at least one graphic representation comprises:
a minimum band related to a minimum peak value of the physiological signal; and
a maximum band related to a maximum peak value of the physiological signal.

10. The method of claim 1, wherein the at least one graphic representation comprises a minimum peak value of the physiological signal superimposed on a maximum peak value of the physiological signal.

11. The method of claim 1, wherein the at least one graphic representation comprises at least one shape indicating the reported FRP.

12. The method of claim 1, further comprising:
receiving patient information; and
adjusting determination of the FRP based on the patient information.

13. The method of claim 12, wherein the patient information comprises one or more of height, weight, body mass index (BMI), body surface area (BSA), hydration level, skin pigmentation, or medication information.

14. The method of claim 1, wherein the physiological signal comprises a blood pressure signal, a plethysmographic (PPG) signal, or a stroke volume signal.

15. The method of claim 1, wherein the reported FRP is the determined FRP.

16. A system for displaying a fluid responsiveness predictor (FRP) based on an analysis of one or more physiological signals of a patient, the system comprising:
an input for receiving a physiological signal responsive to a physiological state of the patient;
a FRP determination module configured to determine an FRP through an analysis of at least one characteristic of the physiological signal over time;
a user interface configured to allow a user to input a request to report the FRP at a requested time;
an FRP reporting module configured to receive the request and instruct the FRP determination module to generate a reported FRP; and
an FRP display module configured to display the reported FRP on a display from the requested time until one of a cease report instruction input through the user interface or an end time for the reported FRP, wherein the reported FRP is displayed via at least one graphic representation.

17. The system of claim 16, wherein the FRP is not displayed if the FRP reporting module does not receive the request.

18. The system of claim 16, wherein the reported FRP is based on a considered time period that extends from an initial time to at least the requested time.

19. The system of claim 16, wherein the FRP determination module is configured to ignore noise within the physiological signal when generating the reported FRP.

20. The system of claim 16, wherein the FRP determination module is configured to generate the reported FRP by averaging the FRP over a considered time period that extends from an initial time to at least the requested time.

21. The system of claim 16, wherein the at least one graphic representation comprises a difference bracket.

22. The system of claim 16, wherein the at least one graphic representation comprises a shaded or colored area between a maximum peak value of a portion of the physiological signal and a minimum peak value of the portion of the physiological signal.

23. The system of claim 16, wherein the at least one graphic representation comprises:
a minimum band related to a minimum peak value of the physiological signal; and
a maximum band related to a maximum peak value of the physiological signal.

24. The system of claim 16, wherein the at least one graphic representation comprises a minimum peak value of the physiological signal superimposed on a maximum peak value of the physiological signal.

25. The system of claim 16, wherein the at least one graphic representation comprises at least one shape indicating the reported FRP.

26. The system of claim 16, wherein the physiological signal comprises a blood pressure signal, a plethysmographic (PPG) signal, or a stroke volume signal.

27. The system of claim 16, wherein the reported FRP is the determined FRP.

28. A method for graphically displaying a predictor of fluid responsiveness of a subject, comprising:
receiving a physiological signal representative of a blood flow characteristic of the subject;
calculating a fluid responsiveness predictor based on modulations of the physiological signal; and
displaying a graphical indication of the fluid responsiveness predictor, wherein the graphical indication includes a representation of an area between portions of the physiological signal.
